(19) United States
(12) Patent Application Publication
(10) Pub. No.: US 2007/0060917 A1
(43) Pub. Date: Mar. 15, 2007

(54) High-efficiency, side-pumped diode laser system

(75) Inventor: Manvel Artyom Andriasyan, San Diego, CA (US)

Correspondence Address: STOUT, UXA, BUYAN & MULLINS LLP, 4 VENTURE, SUITE 300, IRVINE, CA, 92618, US

(73) Assignee: BioLase Technology, Inc.

(21) Appl. No.: 11/595,566

(22) Filed: Nov. 10, 2006

Related U.S. Application Data
(63) Continuation of application No. 10/178,080, filed on Jun. 21, 2002.
(63) Continuation-in-part of application No. 11/033,032, filed on Jan. 11, 2005.

(51) Int. Cl. A61B 18/18

(52) U.S. Cl. 606/10; 606/13

(57) ABSTRACT

An apparatus for cutting or ablating hard tissue, includes an optical cavity; a gain medium disposed within the optical cavity; a diode light pump disposed within the optical cavity and optically aligned to light pump the gain medium to generate laser light. The generated laser light has a wavelength and power density suitable for cutting and ablating hard tissue.

### CROSS-REFERENCE TO RELATED APPLICATIONS

This application is a continuation of co-pending U.S. application Ser. No. 10/178,080, filed Jun. 21, 2002 and entitled HIGH-EFFICIENCY, SIDE-PUMPED DIODE LASER SYSTEM (Att. Docket BI9280P), which is commonly assigned and the contents of which are expressly incorporated herein by reference. This application is also a continuation of co-pending U.S. application Ser. No. 11/033,032, filed Jan. 10, 2005 and entitled ELECTROMAGNETIC ENERGY DISTRIBUTIONS FOR ELECTROMAGNETICALLY INDUCED DISRUPTIVE CUTTING (Att. Docket BI9842P), which is commonly assigned and the contents of which are expressly incorporated herein by reference.

### BACKGROUND OF THE INVENTION

1. Field of the Invention

The present invention relates generally to cutting devices and, more particularly, to diode laser systems.

2. Description of Related Art

A solid-state laser system generally comprises a laser rod for emitting coherent light and a stimulation source for stimulating the laser rod to emit the coherent light. Flashlamps are typically used as stimulation sources. Diodes may also be used for the excitation source. The use of diodes for generating light amplification by stimulated emission is discussed in the book Solid-State Laser Engineering, Fourth Extensively Revised and Updated Edition, by Walter Koechner, published in 1996.

Prior art laser diode pumped lasers have been either end-pumped, as demonstrated in FIG. 1a or side-pumped. End pumping configurations can be more efficient and can produce a better transverse mode. In FIG. 1a, wherein “HR” denotes a high reflectivity element and “OC” denotes an output coupling element, laser output is focused into a fiber via a lens. Side pumping constructions, on the other hand, can be more scalable therefore enabling the generation of relatively high laser power and energy.

### SUMMARY OF THE INVENTION

In accordance with one aspect of the present invention, a method of cutting or ablating hard tissue is disclosed, comprising the steps of providing a gain medium, a diode, and an optical cavity; placing the gain medium and the diode within the optical cavity so that the diode array is optically aligned to pump the gain medium; activating the diode to light pump the gain medium and generate laser light; and directing the laser light onto the hard tissue to cut or ablate the hard tissue.

In accordance with another aspect of the present invention, a method of cutting or ablating hard tissue comprises the steps of providing a gain medium, a diode light pump, and an optical cavity; placing the gain medium and the diode light pump within the optical cavity so that the diode light pump is optically aligned to light pump the gain medium; activating the diode light pump to light pump the gain medium and generate laser light; and directing the laser light onto the hard tissue to cut or ablate the hard tissue.

According to another aspect of the invention, an apparatus for cutting or ablating hard tissue comprises an optical cavity; a gain medium disposed within the optical cavity; a diode light pump disposed within the optical cavity and optically aligned to light pump the gain medium to generate laser light, wherein the generated laser light has a wavelength and power density suitable for cutting and ablating hard tissue.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112.

Any feature or combination of features described herein are included within the scope of the present invention provided that the features included in any such combination are not mutually inconsistent as will be apparent from the context, this specification, and the knowledge of one skilled in the art. In addition, any feature or combination of features may be specifically excluded from any embodiment of the present invention. For purposes of summarizing the present invention, certain aspects, advantages and novel features of the present invention are described. Of course, it is to be understood that not necessarily all such aspects, advantages or features will be embodied in any particular implementation of the present invention. Additional advantages and aspects of the present invention are apparent in the following detailed description and claims.

### BRIEF DESCIRPTION OF THE FIGURES

FIG. 1a is a schematic illustration of an end-pumped diode laser in accordance with the prior art;

FIG. 1b is a side-pumped diode laser according to the present invention;

FIG. 2a is a schematic top view of a laser head according to the present invention;

FIG. 3 is a regulated laser pulse format according to the present invention;

FIG. 4a shows the population inversion in a CW pumping regime according to the present invention;

FIG. 4b shows the resonator Q due to the Q-switch hold-off according to the present invention;

FIG. 4c shows the resulting laser pulse from FIGS. 4a and 4b according to the present invention;

FIG. 5a shows the quasi CW current supplied to the pumping laser diode according to the present invention;

FIG. 5b shows the population inversion in the quasi CW pumping according to the present invention;

FIG. 5c shows resulting laser pulse from FIGS. 5a and 5b according to the present invention;

FIG. 6 is a representation corresponding to a preferred pulse shape; and

FIG. 7 is a close-up view of a pulse of FIG. 6a.

### DETAILED DESCRIPTION OF THE PRESENT INVENTION

Reference will now be made in detail to particular embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims.

In accordance with one aspect of the present invention, a method of cutting or ablating hard tissue is disclosed, comprising the steps of providing a gain medium, a diode array, and an optical cavity; placing the gain medium and the diode array within the optical cavity so that the diode array is optically aligned to side pump the gain medium; activating the diode array to light pump the gain medium and generate laser light; and directing the laser light onto the hard tissue to cut or ablate the hard tissue.

In accordance with another aspect of the present invention, a method of cutting or ablating hard tissue comprises the steps of providing a gain medium, a diode light pump, and an optical cavity; placing the gain medium and the diode light pump within the optical cavity so that the diode light pump is optically aligned to light pump the gain medium; activating the diode light pump to light pump the gain medium and generate laser light; and directing the laser light onto the hard tissue to cut or ablate the hard tissue.

According to another aspect of the invention, an apparatus for cutting or ablating hard tissue, comprises an optical cavity; a gain medium disposed within the optical cavity; a diode light pump disposed within the optical cavity and optically aligned to light pump the gain medium to generate laser light, wherein the generated laser light has a wavelength and power density suitable for cutting and ablating hard tissue.

In any of the above aspects, the gain medium may comprise a laser rod, such as an Erbium-based laser rod. More particularly, the gain medium may comprise an Erbium-based crystalline laser rod for generating laser light in a range between 1.73 and 2.94 microns. The laser light can be generated in the TEMoo mode to overcome thermal effects. In accordance with a method of the present invention, the hard tissue can comprise, for example, tooth or bone tissue. Temporal pulse control can be used to attain a uniform temporal pulse pattern. In another embodiment, gain switching or Q-switching can be used to attain the uniform temporal pulse pattern. The diode light pump can comprise a diode array, and the diode array can be optically aligned to side pump the gain medium. The diode light pump can be placed within the optical cavity so that the diode array is optically aligned to side pump the gain medium.

Any feature or combination of features described herein are included within the scope of the present invention provided that the features included in any such combination are not mutually inconsistent as will be apparent from the context, this specification, and the knowledge of one of ordinary skill in the art.

The methods and apparatuses of this application are intended for use, to the extent the technology is compatible, with existing technologies including the apparatuses and methods disclosed in any of the following patents and patent applications: U.S. Pat. No. 5,741,247; U.S. Pat. No. 5,785,521; U.S. Pat. No. 5,968,037; U.S. Pat. No. 6,086,367; U.S. Pat. No. 6,231,567; and U.S. Ser. No. 09/848,010 (filed May 2, 2001), all of which are assigned to BioLase Technology, Inc. and are incorporated herein by reference.

The diode side pumped Erbium crystalline laser of the present invention may emit at wavelengths between 1.73 and 2.94 μm. The pumping may be accomplished by InGaAs laser diodes configured as bars or arrays emitting at 968 nm, and can be delivered in either a CW (continuous wave) or a QCW (quasi-continuous wave) mode of operation, at power levels that may begin at 40 W. With an optimized output coupling, the light-to-light efficiency can be at least 10% and can reach a magnitude up to 35%. One of the embodiments of this invention is that these efficiency magnitudes are higher than those which may have been previously attained, owing to the inventive design which seeks to maximize the pump-to-laser mode overlap and to optimize outcoupling, specifically tailoring the outcoupling to the pulse format or CW operation of the laser.

The oscillator of the present invention is a plano-plano resonator comprising a high reflectivity mirror and an outcoupling, partially transmitting mirror. For certain applications intracavity elements, such as an electro-optic or acousto-optic cell for Q-switching, or an etalon for wavelength tuning can be introduced. The laser can emit energy in, for example, one of the following modes of operation: CW, gain switched obtained by quasi-CW operation of the pump laser diode, and Q-switched by an acousto-optical (AO) device or Q-switched by an electro-optical (EO) device. Thermal management and temperature control are provided by either air and/or water cooling, with the possibility of using thermo-electric cooling.

In the category of the disclosed diode side pumped lasers included are the following crystals: Er:LiYF₄ (Er:YLF) emitting at 1.73 μm on the Er³⁺⁴I_{13/2} [symbol] ⁴I_{15/2} transition; Er:LiYF₄ emitting at 2.80 μm on the Er³⁺⁴I_{11/2} [symbol] ⁴I_{13/2} transition; Er:Y₃Sc₂GasO₁₂ (Er:YSGG) emitting at 2.79 μm on the Er³⁺⁴I_{11/2} [symbol] ⁴I_{13/2} transition; Er:Gd₃Sc₂GasO₁₂ (Er:GSGG) emitting at 2.8 μm on the Er³⁺⁴I_{11/2} [symbol] ⁴I_{13/2} transition; Er:Gd₃GasO₁₂ (Er:GGG) emitting at 2.82 μm on the Er³⁺⁴I_{11/2} [symbol] ⁴I_{13/2} transition; Er,Tm:Y₃Al₅O₁₂ (TE:YAG) emitting at 2.69 μm on the Er³⁺⁴I_{11/2} [symbol] ⁴I_{13/2} transition; Er:KYF₄ emitting at 2.81 μm on the Er³⁺⁴I_{11/2} [symbol] ⁴I_{13/2} transition; Ho, Yb:KYF₄ emitting at 2.84 μm on the Ho³⁺⁵I₆ [symbol] ⁵I₇ transition; Er:Y₃Al₅O₁₂ (Er:YAG) emitting at 2.94 μm on the Er³⁺⁴I_{11/2} [symbol] ⁴I_{13/2} transition; Er:Y₃AlO₃ (Er:YALO) emitting at 2.71 μm on the Er³⁺⁴I_{11/2} [symbol] ⁴I_{13/2} transition; Er:KGd(WO₄)ₛ (Er:KGW) emitting at 2.8 μm on the Er³⁺⁴I_{11/2} [symbol] ⁴I_{13/2} transition; Er:KY(WO₄)ₛ (Er:KYW); Er:Al₃O₃ emitting on Er³⁺⁴I_{11/2} [symbol] ⁴I_{13/2} transition; Er:Lu₃O₃ emitting at emitting at 2.7 μm on the Er³⁺⁴I_{11/2} [symbol] ⁴I_{13/2} transition; Er:CaF₂ emitting at 2.75-2.85 μm on the Er³⁺⁴I_{11/2} [symbol] ⁴I_{13/2} transition; Cr,Tm,Er:Y₃Al₅O₁₂ (CTE:YAG) emitting at 2.7 μm on the Er³⁺⁴I_{11/2} [symbol] ⁴I_{13/2} transition; Er:BaLu₂F₈ emitting at 2.8 μm on the Er³⁺⁴I_{11/2} [symbol] ⁴I_{13/2} transition; Er:BaY₂F₈ (Er:BYF) emitting at 2.7 μm on the Er³⁺⁴I_{11/2} [symbol] ⁴I_{13/2} transition; and Cr:ZnSe emitting at 2-3 μm.

Due to their efficient interaction with biological tissue and water, these lasers are useful as surgical instruments, in the areas of, for example, dental surgery, orthopedic surgery, tissue ablation, bone cutting and soft tissue surfacing. Particular applications may include use of the laser for expansion of atomized water or fluid particles above a target surface for mechanical cutting or ablation, such as disclosed in U.S. Pat. No. 5,741,247, entitled Atomized Fluid Particles for Electromagnetically Induced Cutting, and U.S. Pat. No. 5,785,521, entitled “Fluid Conditioning System,” the contents of which are expressly incorporated herein by reference.

Another embodiment of the side diode pumped erbium lasers and Ho,Yb:KYF4 laser is that when operated in pulses, the pulsed format is highly repetitive in time and intensity. This performance can facilitate precise and predictable cutting, and can improve cutting efficiency. In dental and medical applications, this feature is consistent with less heat or thermal denaturation of the tissue, which can provide for quicker healing.

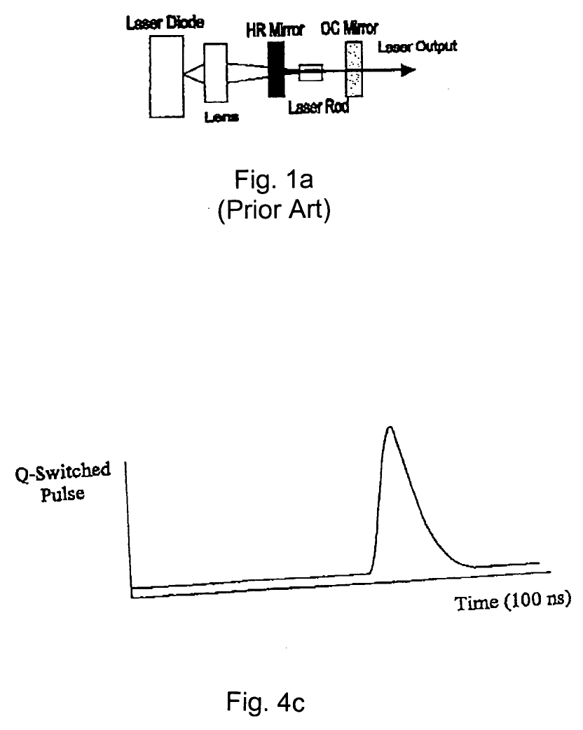

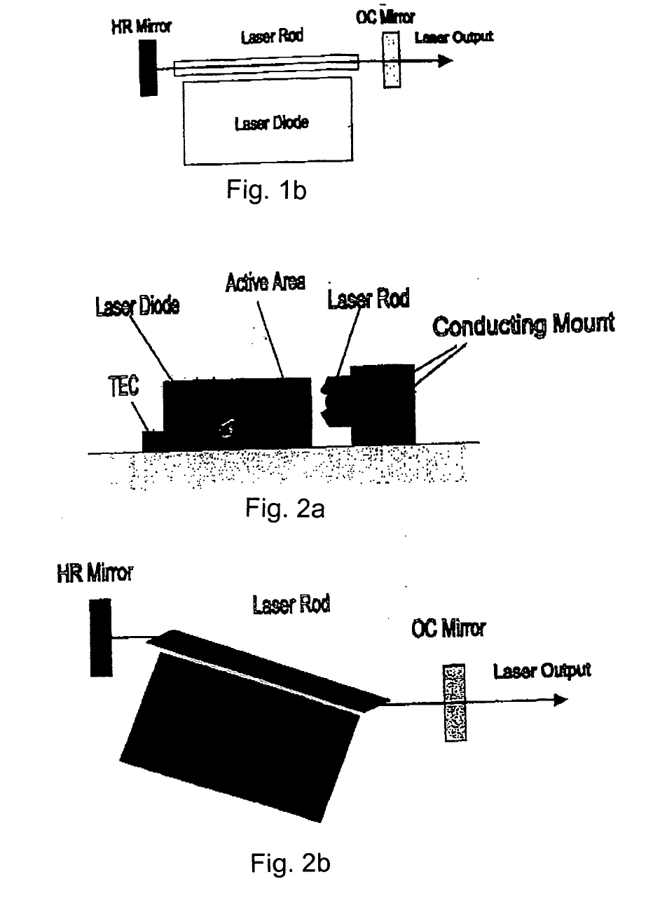

The present invention is configured as shown in Figs. 1a, 2a and 2b. It applies the side-pumped configuration to: 1) pumping of erbium and Ho,Yb:KYF4 crystals to extract laser emission in the 1.73 and 2.94 μm range, 2) dental and medical cutting and resurfacing by mainly the 2.69 to 2.95 μm range, 3) optimization of the dental and medical process by efficient delivery of the laser to the target and minimal thermal process. Configuration of the crystal itself can be rectangular or round. A rectangular shape may be preferred in one embodiment, although a cylindrical shape may function well in modified embodiments. The pumping wavelength should be chosen to be efficiently transferred into the crystal, wherein for example the radiation wavelength of the diode pumping source matches a peak absorption of the active media or crystal. In one embodiment a lens may be used to couple the pump source to the laser rod. Cooling sources and/or lenses may be positioned between the pump source and the laser rod. Regarding FIGS. 2a and 2b, FIG. 2a is a schematic top view of a laser head according to the present invention wherein “TEC” denotes thermo electric cooler, and FIG. 2b is a schematic side view of a laser head according to the present invention wherein opposing ends of the laser rod are cut to the Brewster angle to provide polarization.

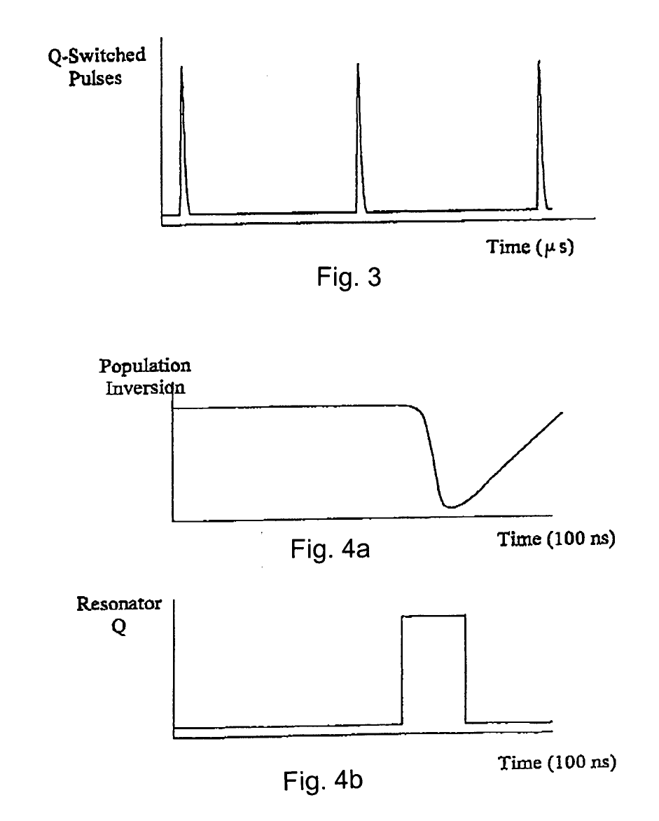

Regarding the present invention's application of the side-pumped configuration to optimize dental and medical processes by efficient delivery of the laser to the target and minimal thermal process, optimization is accomplished by radiating the target with a train of well regulated pulses, as shown in FIG. 3. What is shown is a sequence of narrow pulses, each having a sufficiently high power, for instance 20 kW, and an energy of 8 mJ. With a duty cycle of 0.02% this determines an average power of 4 W. A number of methods may be employed to attain such a pulse format, among them: gain switching and Q-switching by either an electro-optical or an acousto-optical Q-switch.

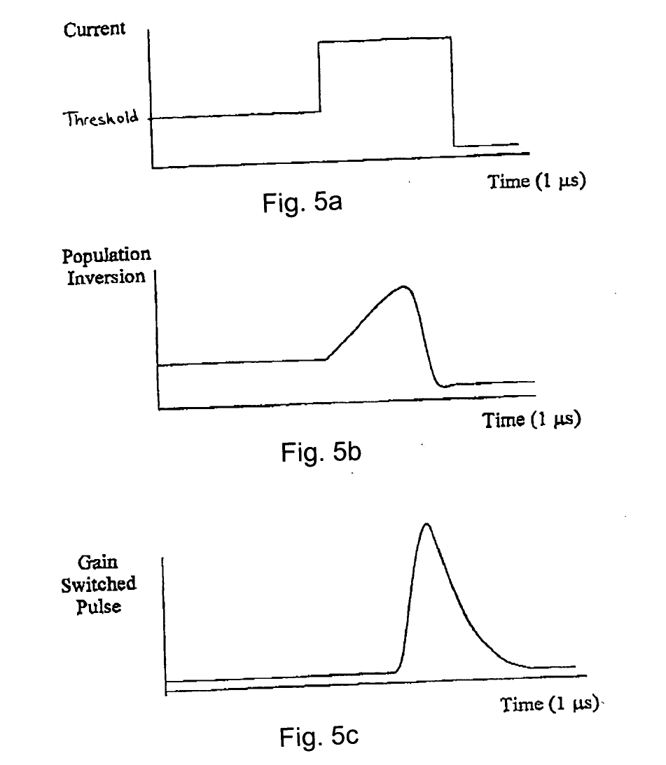

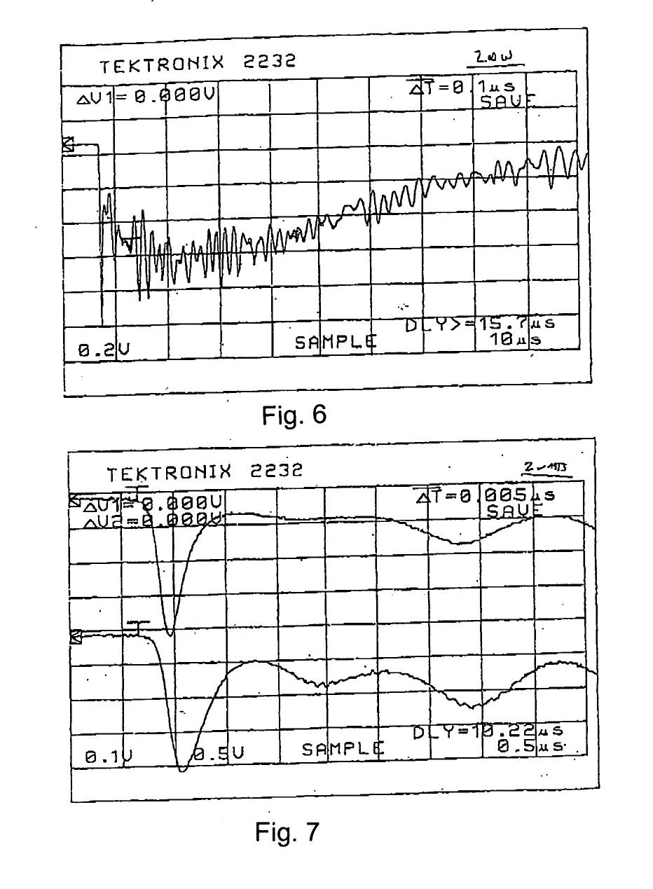

The Q-switch temporal trace is shown in FIGS. 4a-4c, wherein FIG. 4a shows the population inversion in a CW pumping regime, FIG. 4b shows the resonator Q due to the Q-switch hold-off, and FIG. 4c corresponds generally to FIG. 3 and shows the resulting laser pulse. The gain switch temporal trace is shown in FIGS. 5a-5c, wherein FIG. 5a shows the quasi-CW (QCW) current supplied to the pumping laser diode, FIG. 5b shows the population inversion in the QCW pumping regime, and FIG. 5c shows the resulting laser pulse. Because in gain switching the resonator Q is never spoiled, the pulse evolves simultaneously with the buildup of the population inversion. Hence, the dynamics are similar to a free running laser, as in the pulse train shown in FIG. 6. However, as shown in FIG. 5a, the gain is dropped to below threshold once the first spike is generated, thus a gain switch pulse is formed as the first spike only, as shown in FIG. 7. Additional description is provided in the following table.

In view of the foregoing, it will be understood by those skilled in the art that the methods of the present invention can facilitate formation of laser devices, and in particular side-pumped diode laser systems. The above-described embodiments have been provided by way of example, and the present invention is not limited to these examples. Multiple variations and modification to the disclosed embodiments will occur, to the extent not mutually exclusive, to those skilled in the art upon consideration of the foregoing description. Such variations and modifications, however, fall well within the scope of the present invention as set forth in the following claims.

What is claimed is:

1. A method of cutting or ablating hard tissue, comprising the following steps:
activating a diode to light pump a gain medium and generate laser light having a wavelength, pulse format, and power density suitable for cutting and ablating hard tissue and further having a wavelength which is highly absorbed by a fluid;
placing the fluid into a volume in close proximity to the hard tissue; and
directing pulses of the laser light into the volume in close proximity to the hard tissue to cause laser light to be highly absorbed by fluid in the volume and to effectuate the cutting or ablating of the hard tissue.

2. The method as set forth in claim 1, wherein the gain medium comprises a laser rod.

3. The method as set forth in claim 1, wherein the gain medium comprises an Erbium-based laser rod.

4. The method as set forth in claim 1, wherein the hard tissue comprises tooth tissue.

5. The method as set forth in claim 1, wherein the hard tissue comprises bone.

6. The method as set forth in claim 1, wherein the laser light has a wavelength in a range from about 2.69 um to about 2.95 um.

7. A method of cutting or ablating a target, comprising the following steps:
activating a diode to pump a gain medium and generate electromagnetic energy suitable for cutting and ablating a target and which is highly absorbed by a fluid;
placing the fluid into a volume in close proximity to the target; and
directing pulses of the electromagnetic energy into the volume in close proximity to the target to cause electromagnetic energy to be highly absorbed by fluid in the volume and to generate the cutting or ablating of the target.

8. The method as set forth in claim 7, wherein the laser light has a wavelength in a range from about 2.69 um to about 2.95 um.

9. The method as set forth in claim 7, wherein the gain medium comprises a laser rod.

10. The method as set forth in claim 7, wherein the gain medium comprises an Erbium-based laser rod.

11. The method as set forth in claim 7, wherein the target comprises tooth tissue.

12. The method as set forth in claim 7, wherein the target comprises bone.

13. An apparatus for cutting or ablating hard tissue, comprising:
a fluid output configured to place fluid into a volume in close proximity to the hard tissue; and
a diode optically aligned to light pump a gain medium to generate laser light, wherein the generated laser light has a wavelength, pulse, and power density suitable for cutting and ablating hard tissue and further has a wavelength that is highly absorbed by the fluid, the apparatus being configured to direct the laser light into the volume in close proximity to the hard tissue to cause laser light to be highly absorbed by fluid in the volume whereby the hard tissue is cut or ablated.

14. The apparatus as set forth in claim 13, wherein the gain medium comprises a laser rod.

15. The apparatus as set forth in claim 13, wherein the gain medium comprises an Erbium-based laser rod.

16. The apparatus as set forth in claim 13, wherein the generated laser light has a wavelength, pulse, and power density suitable for cutting and ablating tooth tissue.

17. The apparatus as set forth in claim 13, wherein the generated laser light has a wavelength, pulse, and power density suitable for cutting and ablating bone.

18. The apparatus as set forth in claim 13, wherein the gain medium comprises an Erbium-based crystalline laser rod for generating laser light in a range between 1.73 and 2.94 microns.

19. The method as set forth in claim 13, wherein the laser light has a wavelength in a range from about 2.69 um to about 2.95 um.
